(19) United States
(12) Patent Application Publication
(10) Pub. No.: US 2003/0190367 A1
(43) Pub. Date: Oct. 9, 2003

(54) Oxygen enriched implant for orthopedic wounds and method of packaging and use

(76) Inventor: David Balding, Mission Viejo, CA (US)

Correspondence Address: Vic Y. Lin, Myers Dawes Andras & Sherman LLP, Suite 1150, 19900 MacArthur Blvd., Irvine, CA, 92612, US

(21) Appl. No.: 10/410,418

(22) Filed: Apr. 7, 2003

Related U.S. Application Data
(60) Provisional application No. 60/370,076, filed on Apr. 5, 2002.

(51) Int. Cl. A61K 35/32; A61K 31/685

(52) U.S. Cl. 424/549; 514/78

(57) ABSTRACT

For the repair of injured bone or cartilage, an oxygen enriched material is formed with a high-level (partial pressure) and amount (volume) of dissolved oxygen to support tissue healing. The oxygenated material can be placed in a container having an air valve and pressurized in the container with oxygen to supersaturate the material. The resulting container can be sterilized and transported for use. Immediately prior to use, the valve can be operated to vent the pressure in the container thereby facilitating access to the material. The material can then be applied directly to the hard tissue injury. Absorption of the oxygen to facilitate healing can be enhanced with the use of ultrasonic energy. An oxygen inhibiting film can be placed over the material to direct the oxygen toward the wound.

### RELATED APPLICATIONS

This application relates to and claims priority from U.S. Provisional Application No. 60/370,076 filed on Apr. 5, 2002 and entitled “OXYGEN ENRICHED IMPLANT FOR ORTHOPEDIC WOUNDS AND PACKAGING”, the disclosure of which is incorporated herein by reference.

### BACKGROUND OF THE INVENTION

1. Field of the Invention

This invention relates generally to repair of orthopedic wounds, and more specifically to an oxygen enriched implant for use in such repair.

2. Discussion of Related Art

Areas of injured bone, cartilage and/or tissue compromise blood circulation, reducing the oxygen available to the surrounding tissue. Injuries may commonly result from a traumatic accident, surgery to correct trauma or degenerative processes. Ironically, the surgery that is often performed to correct an earlier traumatic injury can also perpetuate the injury. Both the traumatic injury and the resulting surgery inevitably cut through capillaries, arterioles and vennules. The reduced blood flow results in insufficient oxygen to fully support the metabolic needs of the tissues. Cell death, atrophy and osteonecrosis are induced by lack of oxygen.

Paradoxically, injured tissue has a particularly high need for oxygen to support the healing process. The early stages in bone healing involve lymphocytes and osteoclasts which use considerable oxygen as they resorb damaged or un-needed tissues in preparation for the growth of new bone and associated tissues. Lack of oxygen delays the onset of the healing and bone formation process and slows healing once it is in progress. Additionally, low oxygen levels may increase the potential for infection or prolong existing infection.

Surgical bone repairs are often accomplished with the use of bone void filler materials used to fill space and promote the body's natural healing and bone formation processes. The healing processes in the days following injury and surgical repair include the ingrowth of blood vessels and the restoration of the oxygen supply. However, until the body has reestablished a blood supply, the injured tissue suffers from acute lack of oxygen.

Therefore, currently used bone void filler materials and other orthopedic implants and materials do not address the acute lack of oxygen at injured bone and cartilage and adjacent tissue.

The benefits of oxygen for wound healing are conclusively demonstrated by the use of hyperbaric therapy for tissue and bone wound therapy. The patient is placed in a chamber with elevated pressure and the resulting increase in the oxygen level promotes the diffusion of oxygen into oxygen starved tissues. Collagen formation and capillary ingrowth is promoted by elevated oxygen levels. These-processes allow closure of defects and an enhanced vascular bed for healing. However, there are major disadvantages for routine use of hyperbaric therapy. The patient must be transported to specialized facilities and spend hours or days confined in chambers under close supervision of certified hyperbaric physicians and therapists. The patient must not be claustrophobic or intolerant of pressure changes. Although the efficacy of hyperbaric therapy for orthopedic wound healing is well demonstrated, the aforementioned disadvantages have limited its use to only selected patients.

Oxygen levels can be elevated in a patient by having the patient breathe pure oxygen or air with increased oxygen. Unlike a hyperbaric chamber, the partial pressure of oxygen is limited to one atmosphere. Breathing oxygen gas raises the oxygen level in the blood. However, orthopedic wound areas have disrupted blood circulation. Acute benefits of breathing oxygen would be limited to slow diffusion into anoxic wound areas from adjacent perfused areas. Therefore, the larger orthopedic wounds most in need of oxygen are least likely to have significant increase in oxygen from breathing oxygen. There are well known risks associated with the use of oxygen for breathing. These risks include increased fire hazard, lung damage, seizures, myopia and optic neuritis as a rare contraindication.

In concept, oxygen levels in poorly perfused tissue could be increased by administering oxygen carrying “artificial blood” into the blood of a patient with orthopedic wounds. Any benefits, if any, would require that the patient also breathe supplemental oxygen and be subject to the risks of breathing oxygen as well as loading the circulatory system with an artificial material. Artificial blood does not directly address lack of oxygen at a localized orthopedic wound.

Oxygen supersaturated emulsions, suspensions and gels are described for use in treating localized areas of soft tissue (Spears, Sep. 1, 1998, U.S. Pat. No. 6,169.117) Oxygen saturated emulsions, suspensions and gels are described per use in treating localized areas of soft tissue. However, there is no contemplation that these compositions can be used for hard tissue such as bone and cartilage. In soft tissue injuries, repair is greatly facilitated by vascular in growth which can carry oxygenated blood to the wound site. In the absence of this high level of circulation, for example in hard tissues, impaired vasculature cannot offer this advantage.

Conventional packaging for pharmaceutical or medical devices (e.g. cans, jars, paper or polymeric pouches etc.) is not suitable for oxygen supersaturated emulsions, gels or solutions. Supersaturated materials are loaded with oxygen at pressures well above (0.5 kbar to 1.0 kbar) ambient pressure and release oxygen when they are at ambient pressure. Storage at 10 to 150 bar range is needed to maintain useful levels of oxygen and prevent outgassing. Simply maintaining pressure is not sufficient. Supersaturated materials may rapidly outgas or bubble or even foam if rapidly depressurized. Accordingly suitable packaging is needed for the storage, transport and use of supersaturated emulsions, suspensions and gels used for treating tissues.

### SUMMARY OF THE INVENTION

Certain preferred embodiments of the present invention, an oxygen enriched material is combined with filler materials and adapted for use in orthopedic wounds. The oxygen enriched material is packaged in containers that have provisions for maintaining high pressure and controlled decrease in pressure prior to opening the package for treatment of the patient.

In accordance with the present invention, an oxygen supply material is provided with a high level (partial pressure) and amount (volume) of dissolved oxygen to support tissue healing and bone formation at an orthopedic wound site. This occurs prior to vascular ingrowth and is accomplished with materials that can be sterilized, shaped, textured and molded in a manner suitable for filling bone voids. This is accomplished without supplemental breathing oxygen, hyperbaric therapy, or artificial blood. The oxygen supply material can be supersaturated in a pressurized package and provided with appropriate valving to reduce package pressure prior to opening.

These and other features and advantages of the invention will become more apparent with the description of preferred embodiments in reference to the associated drawings.

### DESCRIPTION OF THE DRAWINGS

FIG. 1 is a schematic view illustrating formulation of an oxygen supply material associated with the present invention;

FIG. 2 is a schematic view illustrating formation of the material to a convenient shape, using a piston for example;

FIG. 3 is a side view of a pressurized container adapted to receive and further process the oxygen supply material;

FIG. 4 is the side elevation view of the container being pressurized with oxygen to supersaturate the oxygen supply material;

FIG. 5 is a side elevation view of the container and the supersaturated oxygen supply material being sterilized;

FIG. 6 is a side elevation view of the container illustrating a step wherein the container is vented or depressurized;

FIG. 7 is a side elevation view of the container showing the lid removed following venting to provide access to the supersaturated oxygen supply material;

FIG. 8 is a side elevation view partially in section showing a high strength support for receiving the container during pressurization;

FIG. 9 is a schematic view of a bone wound impacted with the oxygen supply material and being energized with ultrasonic energy; and

FIG. 10 is a side elevation view of the bone wound and impacted oxygen supply material covered by an oxygen impermeable film which directs the oxygen of the supply material into the bone wound.

### DETAILED DESCRIPTION OF THE PREFERRED EMBODIMENTS AND BEST MODE OF THE INVENTION

There are various possibilities for the combination of an oxygen supply material with implants for orthopedic wounds. The combinations can be varied so long as the resulting implant has shaping and texture properties that are workable for the surgeon to fill the bone void, and the resulting implant functions as a scaffold for bone growth and healing of the orthopedic wound. One skilled in the art of bone implant and bone filling materials can anticipate a variety of possible combinations once the concept of including an oxygen supply material is appreciated. The following are examples of bone void filling materials that could be used in combination with the oxygen supply material:

Demineralized bone;

Demineralized bone in a glycerol carrier;

Calcium salts in granular or paste form (e.g. calcium sulfate, calcium phosphate, calcium carbonate);

Calcium salts and a phospholipid carrier;

Calcium salts and a platelet rich gel; and

Bone chips (autograft or allograft bone).

As can be seen, there are a wide variety of materials that can be combined with an oxygen supply material and thereby benefit the patient from the delivery of oxygen to the orthopedic wound site for accelerated wound healing.

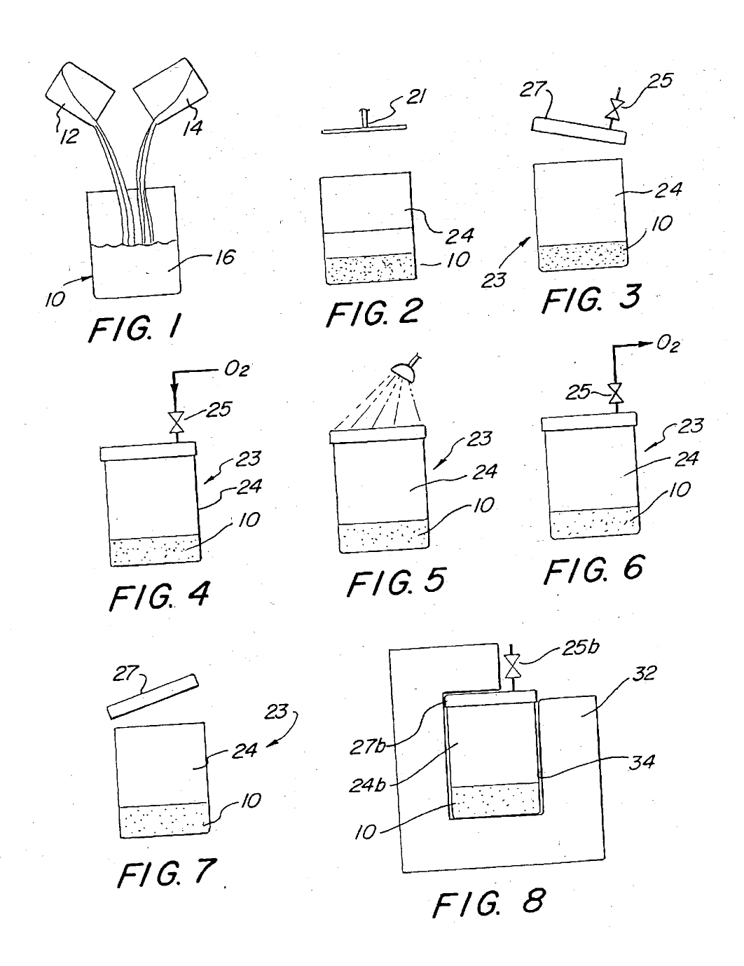

A bone filling material 10 is illustrated in FIG. 1 and designated by the reference numeral 10. As an example and not by way of limitation, the bone filling material 10 may be formed by combining perfluoronated hydrocarbon (PFC) 12, phospholipids 14 and demineralized bone matrix 16. As discussed above, a variety of materials may be used and different combinations may be employed to form the bone filling material. The elements 12, 14, 16 are mixed to a “putty”-like consistency and then molded by a piston 21, for example as illustrated in FIG. 2. The bone filling material 10 may comprise a gel, emulsion, or solution, and is preferably moldable. Once the material 10 has been prepared it can then be disposed in a package or container 23 suitable to hold pressure in the manner described below. In a preferred embodiment, the container 23 is composed of a jar with 24 with a removable lid, 27 having a port or valve 25 as illustrated in FIG. 3.

Pressurized oxygen can be introduced into the container 23 through the valve 25 as shown in FIG. 4. Additional oxygen may be added over time as the oxygen within the container 23 becomes absorbed into the bone filling material 10. The container 23, the bone filling material 10, and the oxygen within can then be sterilized (FIG. 5), stored and ultimately transported to any site for use.

In order to access the oxygenated material 10, the valve 25 can be opened, as illustrated in FIG. 6, to release the pressure within the container 23. Once the pressure is released, the container 23 may be opened by removing the lid 27 to access the oxygenated material as shown in FIG. 7. The material 10 can then be applied to a particular injured area of the patient, providing the necessary oxygen to facilitate faster healing.

FIG. 8 illustrates a second preferred embodiment of a package wherein elements of structure similar to those previously discussed are designated by the same reference numeral followed by the letter “b.” The package 30 comprises a container 23b having a jar 24b and a lid 27b with a valve 25b. The bone filling material 10, which may be previously saturated, is placed into the container 23b. The lid 27b is then closed. Prior to pressuring the container 23b, however, the container 23b is placed within a larger support structure, or enclosure 32, configured to receive the container 23b. In a preferred embodiment, the enclosure 32 includes inner surfaces 34 that are adapted to abut the outer surfaces of the container 23b.

The container 23b is then pressurized by introducing oxygen through the valve 25b. It will be appreciated that the enclosure 32 provides additional support to the container 23b, thereby enabling the container 23b to hold a higher pressure. The entire package may be shipped to the treatment site. Alternatively, pressure within the container 23b may be decreased to a level which the container 23b can sustain by itself. The container 23b may then be removed from the enclosure 32 and then shipped to the treatment site. In the manner previously discussed the pressure can be further reduced by venting the oxygen within the container 23b prior to opening the lid 27b and removing the material 10.

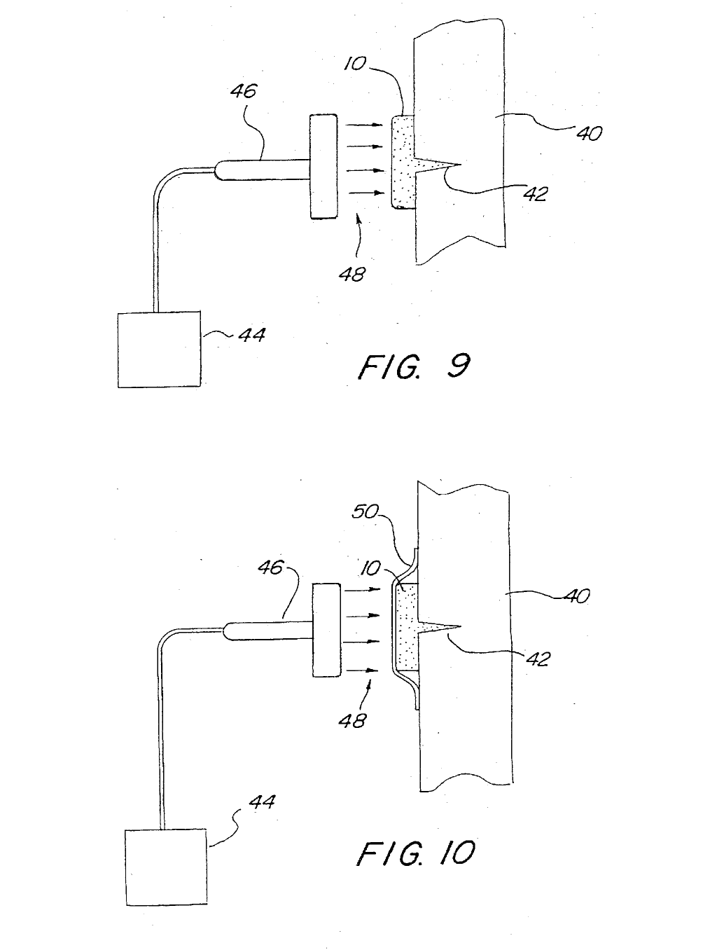

A preferred method for using the bone filling material 10 is illustrated in FIG. 9. In this view, a bone 40 has a wound 41 which is impregnated with the bone filling material 10. Ultrasonic energies supplied by a source 44 is delivered to a hand piece 46 with this apparatus, ultrasonic energy 48 can be directed toward the filling material 10 and associated bone wound 42. By directing ultrasonic energy through the supersaturated oxygen filling material 10, diffusion of the oxygen into the bone wound 42 can be accelerated. It is contemplated that this process can be used in an open wound wherein the bone wound 42 is accessible, or in a closed wound wherein the ultrasonic energy 48 would be directed through the skin of the patient.

Any similar method illustrated in FIG. 10, the bone filler material 10 is applied to the bone wound 42 in the manner previously described. However, in this case, an oxygen inhibiting or impermeable material 50 can be placed over the bone filling material 10. In this manner, the oxygen inhibiting material 50 functions to direct the oxygen of the material 10 toward the wound site 42. This prevents the oxygen from being lost to atmosphere or to locations other than the wound site 42. The oxygen inhibiting material 50 can be applied as a coating, but in a preferred embodiment it is applied as a film to cover the bone filling material 10. The ultrasonic energy 48 can be applied in the manner previously discussed and directed through the oxygen inhibiting material 50 toward the bone filling material 10 and associated wound site 42.

Many alterations and modifications may be made by those having ordinary skill in the art without departing from the spirit and scope of the invention. Therefore, it must be understood that the illustrated embodiments have been set forth only for the purposes of examples and that they should not be taken as limiting the invention.

The words used in this specification to describe the invention and its various embodiments are to be understood not only in the sense of their commonly defined meanings, but to include by special definition in this specification the generic structure, material or acts of which they represent a single species.

What is claimed is:

1. A composition for repairing bone injuries, comprising:
a bone void filling material; and
an oxygen supply material saturating the bone void filling material.

2. The composition recited in claim 1, wherein the oxygen supply material comprises a perfluoronated hydrocarbon.

3. The composition recited in claim 2, wherein the oxygen supply material comprises a phospholipid.

4. The composition recited in claim 3, wherein the bone void filling material comprises a demineralized bone matrix.

5. The composition recited in claim 1,. wherein the oxygen supply material supersaturates the bone void filling material.

6. A method for packaging an oxygen saturated composition, comprising the steps of:
placing the composition in a container having a lid for sealing the container;
pressurizing the container with oxygen;
storing the pressurized container for ultimate surgical use;
venting the pressurized oxygen from the container prior to use; and
removing the lid from the container to access the composition.

7. The method recited in claim 6, wherein the pressurizing step includes the steps of:
providing a valve communicating with regions interior of the container; and
introducing oxygen under pressure through the valve and into the container to pressurize the composition in the container.

8. The method recited in claim 7, wherein the venting step includes the step of withdrawing the pressurized oxygen through the valve.

9. The method recited in claim 7, wherein the providing step includes the step of mounting the valve in the lid of the container.

10. The method recited in claim 7, wherein the pressurizing step includes the step of reinforcing the container during the introducing step.

11 A method for repairing a hard tissue injury in a patient, comprising the steps of:
providing an oxygen saturated void filling composition;
placing the composition in proximity to the injury to provide a supply of oxygen to the injury thereby facilitating healing of the injury.

12. The method recited in claim 11, wherein the hard tissue includes at least one of bone and cartilage.

13. The method recited in claim 12, further comprising the step of:
applying ultrasonic energy to the hard tissue injury.

14. The method recited in claim 13, wherein the hard tissue injury is covered by skin of the patient, and the applying step includes the step of:
introducing ultrasonic energy through the skin of the patient to the hard tissue injury.

15. The method recited in claim 11, wherein the placing step includes at least one of spraying the composition on the injury, coating the composition on the injury; and molding the composition into the injury.

16. The method recited in claim 11, further comprising the step of:
covering the injury and the composition with a layer of oxygen inhibiting material.

17 The method recited in claim 16, wherein the oxygen inhibiting material is impermeable to oxygen.

18. The method recited in claim 13, further comprising the step of:
covering the injury and the composition with a layer of oxygen inhibiting material; and
introducing the ultrasonic energy through the film and into the composition.

19. The method recited in claim 14, wherein the ultrasonic energy is applied periodically.
